(19) United States
(12) Patent Application Publication
(10) Pub. No.: US 2009/0060121 A1
(43) Pub. Date: Mar. 5, 2009

(54) COMPUTED TOMOGRAPHY DATA ACQUISITION APPARATUS AND METHOD

(75) Inventors: Andy Ziegler, Hamburg (DE); Tim Nielsen, Hamburg (DE)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N. V., Eindhoven (NL)

(21) Appl. No.: 12/293,087

(22) PCT Filed: Mar. 5, 2007
(86) PCT No.: PCT/US07/63279

§ 371 Date: Sep. 16, 2008

Related U.S. Application Data
(60) Provisional application No. 60/767,300, filed on Mar. 16, 2006.

(51) Int. Cl. A61B 6/00

(52) U.S. Cl. 378/8

(57) ABSTRACT

A computed tomography scanner includes a first (20) and a second (21) detector. The second detector (21) has a relatively higher spatial resolution and a relatively smaller field of view (204) than that of the first detector (20). Projection data generated by the detectors (20, 21) is combined and reconstructed so as to generate relatively high resolution volumetric data (318) indicative of a region of interest (314) in an object under examination.

The present application is directed to the radiographic imaging, and particularly to techniques for reducing the effects of truncation artifacts resulting from detectors having a relatively limited field of view. It finds particular application to x-ray computed tomography (CT), and especially in situations where it is desirable to produce high resolution images of a limited region of interest (ROI).

CT scanners have proven to be invaluable in providing information indicative of the internal structure of an object. In medical imaging, for example, CT scanners are widely used to provide images and other information about the physiology of human patients. Typically, the information generated by a CT scan is reconstructed to generate volumetric data which is in turn presented by way of one or more human readable images.

Recent trends have seen the rapid adoption of multi-slice CT as well as a move to systems having an ever faster rotation speeds. As a result, CT scanners have made increasing inroads in cardiac applications, which typically benefit from improved spatial and temporal resolutions.

Commercially available CT systems traditionally include a generally arcuate radiation sensitive detector. To avoid truncation artifacts in the reconstructed volumetric data, the detector should have a transaxial field of view which is larger than the transaxial dimensions of the objects to be imaged. While these detectors have proven useful in a wide variety of applications, technical and economic considerations typically limit the available spatial resolution.

Flat panel detectors have also been detector. Such detectors typically have a relatively higher resolution than traditional CT detectors. However, technical and economic considerations typically limit the physical size of the detector and thus the available field of view. As a result, flat panel detectors are ordinarily more suitable for use in imaging relatively small objects. While such detectors can be used to image relatively larger objects, the resultant truncation artifacts have offset the benefits provided by the increased spatial resolution. This is especially true in cardiac imaging and other applications which require a relatively high spatial resolution over a relatively small field of view.

Aspects of the present invention address these matters, and others.

According to one aspect of the present invention, a tomographic apparatus includes a first radiation sensitive detector which generates first projection data indicative of an object disposed in an examination region, a second radiation sensitive detector which generates second projection data indicative of the object. The second detector has a second transaxial field of view which is smaller than a transaxial dimension of the object, such that volumetric data reconstructed using the second projection data would contain truncation artifacts. The apparatus also includes means for correcting the second projection data so as to reduce the truncation artifacts, wherein the correction is a function of the first projection data. The apparatus also includes a corrected data reconstructor which generates volumetric data indicative of the corrected second projection data.

According to another aspect, a tomography method includes receiving first projection data generated by a first radiation sensitive detector and receiving second projection data generated by a second radiation sensitive detector. The projection data is indicative of an interior of an object, the object has a transaxial dimension the second projection data is indicative of the interior of the object, the second detector has a second transaxial field of view, and the second transaxial view is smaller than a transaxial dimension of the object, whereby volumetric data reconstructed using the second projection data would contain truncation artifacts. The method also includes correcting the second projection data so as to reduce the truncation artifacts as a function of the first projection data, reconstructing the corrected second projection data, and generating a human readable image indicative of the reconstructed data.

According to another aspect of the invention, a computer readable storage medium contains instructions which, when executed by a computer, cause the computer to carry out a method for reducing truncation artifacts resulting from the tomographic reconstruction of projection data acquired using a first x-ray detector having a first transaxial field of view less than a transaxial dimension of an object under examination. The projection data includes projections which include a contribution from a portion of the object located inside the transaxial field of view and a contribution from a portion of the object located outside the transaxial field of view. The method includes using first volumetric data indicative of a measured radiation attenuation of the object to modify a projection so as to reduce a contribution to the projection from a portion of the object located outside the transaxial field of view, repeating the step of using volumetric data for each of a plurality of projections, and reconstructing the modified projections to generate second volumetric data indicative of the radiation attenuation of the object.

According to another aspect, a computed tomography apparatus includes a first x-ray source, a first x-ray detector which receives x-ray generated by the first x-ray source and which have traversed an examination region, a second x-ray source, and a second x-ray detector which generates a plurality of projections indicative of x-rays generated by the second x-ray source and which have traversed the examination region. The second x-ray detector has a second transaxial field of view and a second transaxial resolution. The first transaxial field of view is larger than the second transaxial field of view and the first transaxial resolution is less than the second transaxial resolution. The apparatus also includes a first reconstructor operatively connected to the first x-ray sensitive detector and adapted to generate first volumetric data, an ROI filter which filters an ROI from the first volumetric data, a projection calculator which calculates projections through the filtered first volumetric data, a projection data subtractor which subtracts the calculated projections from spatially corresponding projections from the second x-ray detector and a data reconstructor which generates volumetric data indicative of the subtracted projection data.

Those skilled in the art will appreciate still other aspects of the present invention upon reading and understanding the attached figures and description.

The present invention is illustrated by way of example and not limitation in the figures of the accompanying drawings, in which like references indicate similar elements and in which:

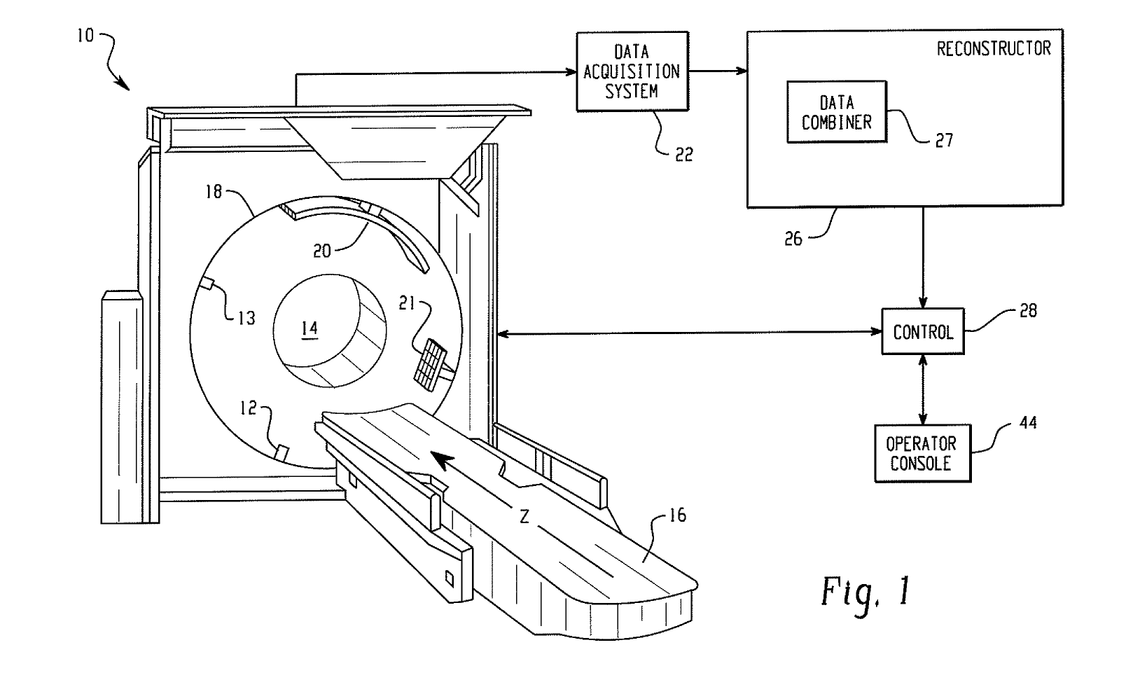

FIG. 1 depicts a CT scanner.

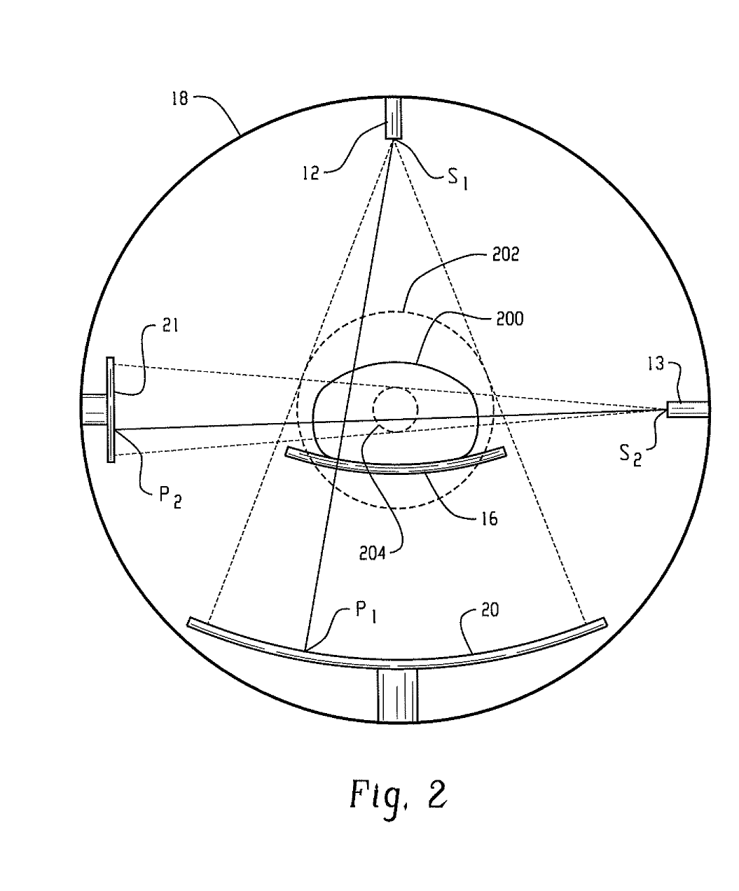

FIG. 2 depicts the acquisition geometry of a CT scanner.

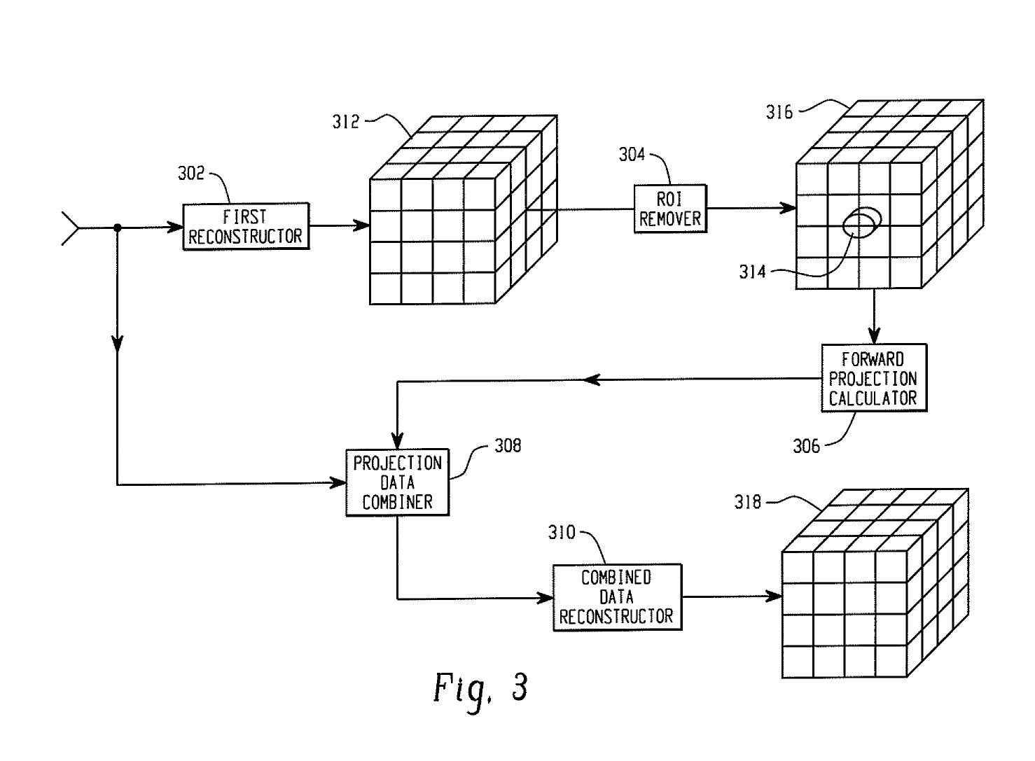

FIG. 3 is a functional block diagram of a data combiner.

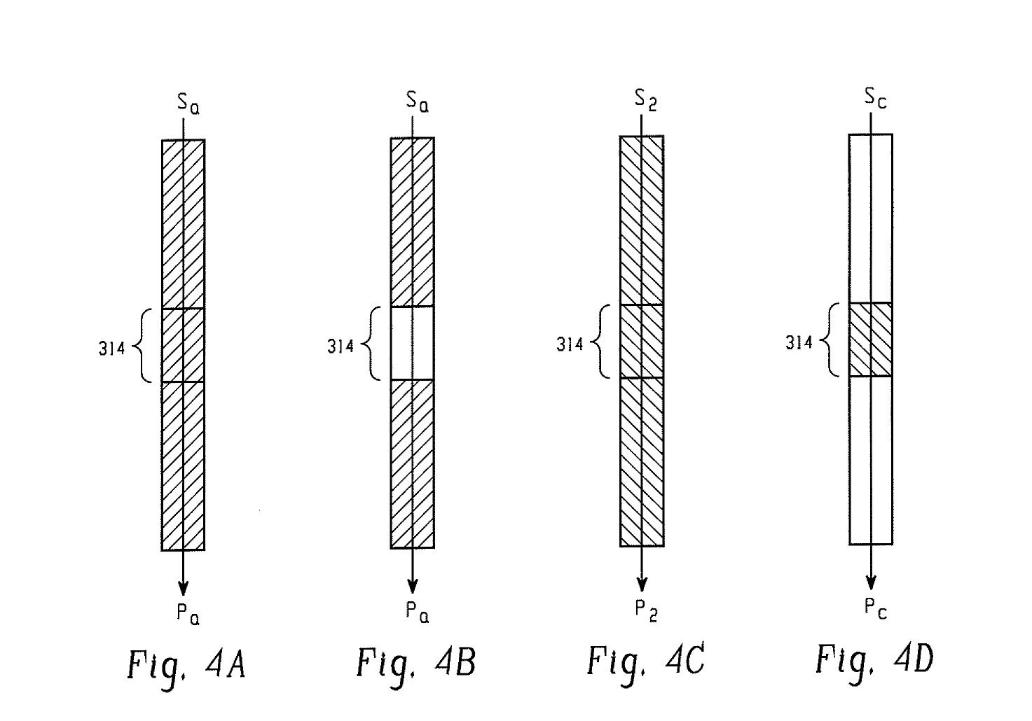

FIGS. 4a, 4b, 4c, 4d depict projection data.

FIG. 5 depicts a technique for using scan data to generating a human readable image.

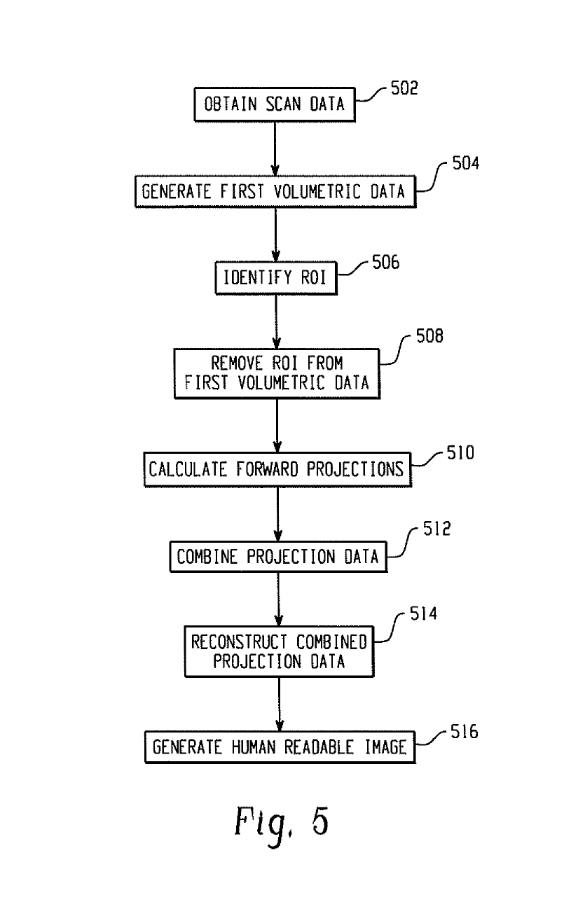

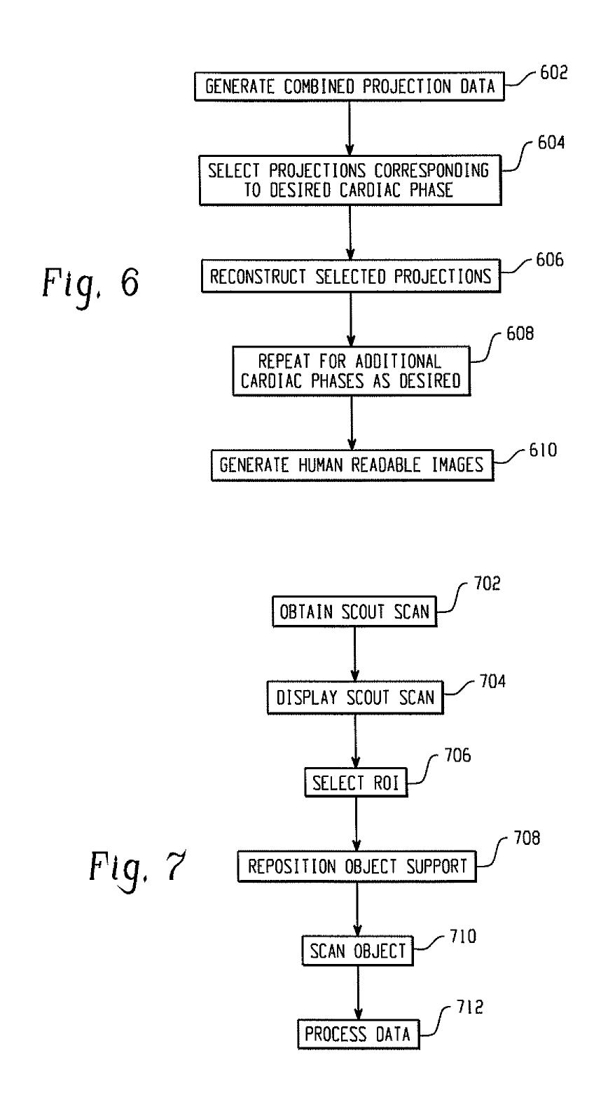

FIG. 6 depicts a technique for using scan data to generate a human readable image having an improved temporal resolution.

FIG. 7 depicts a technique for interactively selecting a region of interest.

With reference to FIG. 1, a CT scanner 10 includes a rotating gantry 18 which rotates about an examination region 14. The gantry 18 supports a first radiation source 12 such as an x-ray tube and a first x-ray sensitive detector 20 which subtends an arc on the opposite side of the examination region 14. The gantry 18 also supports a second x-ray source 13 and a second x-ray sensitive detector 21. X-rays produced by the x-ray sources 12, 13 traverse the examination region 14 and are detected by the detectors 20, 21. The detectors 20, 21 in turn generate respective first and second projection data indicative of the detected radiation.

The first detector 20 is characterized by a relatively low transaxial resolution and a relatively large transaxial field of view. In one implementation, the detector includes an arcuate array of detector elements 100 arranged in a plurality of longitudinal rows or slices and transverse columns. In one implementation, the detector includes 64 or more slices. Each detector element 100 includes a scintillator in optical communication with a photodiode. The photodiodes are preferably fabricated from arrays of back illuminated photodiodes (BIPs), although other photodiode or photodetector technologies can be used. A so-called fourth generation scanner configuration, in which the detector 20 spans an arc of 360 degrees and remains stationary while the x-ray source 12 rotates, as well as flat panel detectors, may also be implemented. Detector having greater or lesser number of slices may likewise be implemented. Depending on the configuration of the detector 20, the first x-ray source 12 generates a beam of radiation having a generally conical, fan, or other desired shape.

The second detector 21 is characterized by a transaxial spatial resolution which is higher and a transaxial field of view which is smaller than that of the first detector 20. The second detector 21 may be implemented as a flat panel detector arranged as a two-dimensional, n×m array of detector elements, although other implementations are possible. For example, the second detector may be implemented in an arcuate array of detector elements similar to those of the first detector 20, but having a relatively higher resolution and having a desired longitudinal extent. The second x-ray source 13 likewise generates a radiation beam consistent with the configuration of the second detector 21.

A data acquisition system 22 preferably located on the rotating gantry 18 receives the projection data generated by the detectors 20, 21 and provides necessary signal conditioning, analog to digital conversion, multiplexing, and like functionality. While illustrated as a single data acquisition system, separate data acquisition systems may be provided for the first and second detectors 20, 21.

A reconstructor 26 reconstructs the data from the detectors 20, 21 to generate volumetric data indicative of radiation attenuation of the object under examination, for example the interior anatomy of a human patient. As will be described in further detail 26, the reconstructor 26 includes a data combiner 27 which uses the data acquired by the lower resolution, larger field of view first detector 20 and the higher resolution, smaller field of view second detector 21 so as to generate a relatively high quality image of a region of interest (ROT) of the object.

A general purpose computer serves an operator console 44. The console 44 includes a human-readable output device such as a monitor or display and an input device such as a keyboard and mouse. Software resident on the console allows the operator to control the operation of the scanner by establishing desired scan protocols, initiating and terminating scans, viewing and otherwise manipulating the volumetric image data, and otherwise interacting with the scanner.

An object support 16 supports an object such as human patient in the examination region 14. The support 16 preferably includes drives which move the support 16 to facilitate the positioning of region(s) of interest of the object in the detector 20, 21 fields of view. The support is also moved in coordination with the rotation of the gantry 18 so as to provide a helical, circular or other desired scanning trajectory.

A controller 28 coordinates the various scan parameters as necessary to carry out a desired scan protocol, including x-ray source 12, 13 parameters, movement of the patient couch 16, and operation of the data acquisition system 26.

Turning now to FIG. 2, the acquisition geometry is shown in greater detail for a given rotating gantry 18 position and object under examination 200. The first detector 20 has a transaxial field of view 202 which is preferably equal to or greater than the maximum transaxial dimension of the object 200. The second detector 21 has a transaxial field of view 204 which is smaller than that of the first detector. Depending on the size of the object 200, the extent of the second detector 21 field of view may also be less than the transaxial extent of the object 200. As will be appreciated by those skilled in the art, volumetric data reconstructed using only the data generated by the second detector would then contain truncation artifacts.

An exemplary projection acquired by the first detector 20 is denoted by the line P₁-S₁, where P₁ represents the position of an exemplary detector element of the first detector 20, and S₁ represents the position of the position of the first x-ray source 12. Similarly, an exemplary projection acquired by the second detector 21 is denoted by the line P₂-S₂, where P₂ represents the position of an exemplary detector element of the second detector 21, and S₂ represents the position of the position of the second x-ray source 13. As the detectors 20, 21 each include a plurality of detector elements, data acquired at each gantry 18 position includes a plurality of projections. Rotation of the gantry 18 and movement of the object support 16 are coordinated so that the detectors 20, 21 traverse a circular, helical, or other desired trajectory about the object 200, thus generating projection data at each of a plurality of positions.

FIG. 3 is a functional block diagram of the data combiner 27, which in the illustrated embodiment includes a first reconstructor 302, an ROI filter or remover 304, a forward projection calculator 306, a projection data combiner 308, and a combined data reconstructor 310.

The first reconstructor 302 reconstructs data acquired by the first detector 20 to generate first volumetric data 312 indicative of the object. The reconstruction is typically performed using filtered back projection techniques as are well known to those skilled in the art, although iterative or other suitable reconstruction techniques may also be implemented. As the first volumetric data 312 will subsequently be used to approximate the line integrals of projections outside the field of view of the second detector 21, the first volumetric data 312 may be of lower quality than that generated in a typical diagnostic scan. For example, the reconstruction parameters may be established so that the volumetric data 312 is of a relatively low resolution. The scan parameters may also be selected to produce a relatively low dose, and hence relatively noisier, volumetric data 312. Of course, the scan and reconstruction parameters may be selected so that the volumetric data 312 is of diagnostic quality.

The ROI remover or filter 304 removes an ROI 314 from the volumetric data 312 so as to generate filtered volumetric data 316. In one implementation, the ROI 314 is selected by the user. In such an implementation, the volumetric data 312 may be advantageously displayed on the operator console 44, and the user selects the desired ROI 314 using the mouse and/or keyboard. In another, the ROI 314 determined automatically or semi-automatically using a suitable image processing technique such as segmentation. A particular advantage of such a technique is that the ROI may be selected so as to exclude strong absorption gradients, as may occur when both soft tissue and bone are present in the ROI. The use of relatively fast, approximate cone beam reconstruction techniques, which are typically sensitive to such gradients, is thus facilitated. Moreover, the impact of missing data may also be reduced, particularly in axial reconstructions in which strong absorption gradients can lead to undesirable image artifacts. In still another implementation, the ROI 314 is established to be coextensive with the field of view 204 of the second detector 21. In any case, however, the ROI 314 is preferably located so as to fall within the field of view 204 of the second detector 21.

Voxels falling within the region of interest 314 are filtered or removed from the volumetric data 312, for example by setting them to the value of air (e.g., −1000 HU). To avoid discontinuities in the data, an interpolation or smoothing operation may be performed on voxels near the interface between the ROI 314 and the remaining volumetric data 316.

The forward projection calculator 306 calculates projections through the modified volumetric data 316 corresponding to the second detector 21 trajectory. More particularly, the line integrals through the modified volumetric data 316 are calculated for projections corresponding to those generated by the second detector 21. As the projections do not correspond to the coordinate system of the modified data 316, the projections may be calculated using a high order interpolation technique based on voxels in the neighborhood of the projections.

The projection data combiner 308 combines the data generated by the forward projection calculator 306 with the projection data generated by the second detector 21. More particularly, the various projections generated by the second detector 21 are subtracted from spatially corresponding projections generated by the projection calculator 306.

The subtraction process for an exemplary projection is illustrated in FIG. 4. FIG. 4a depicts an arbitrary projection along path Sₐ-Pₐ through the volumetric data 312. The projection includes attenuation contributions from both inside and outside the region of interest 314 and the field of view 204 of the second detector. FIG. 4b depicts the arbitrary projection Sₐ-Pₐ with the contribution from voxels in the region of interest 314 being filtered or removed for processing by the forward projection calculator 306. FIG. 4c depicts the projection S₂-P₂ acquired by the second detector 21 along the arbitrary projection Sₐ-Pₐ. The projection, which corresponds to the line integral of the radiation attenuation along the projection S₂-P₂, includes attenuation contributions from both inside and outside the region of interest 314. FIG. 4d depicts the projection data as generated by the combiner 308. As can be seen, the attenuation contributions from outside the region of interest are largely cancelled, so that the projection data is indicative primarily of the radiation attenuation in the ROI 314, and hence in the field of view 204 of the second detector 21. Note that the resolution of the first volumetric data, the spatial correspondence between the measured and calculated projections will affect the accuracy of completeness of the cancellation. In any case, truncation artifacts which would ordinarily be generated by the reconstruction of the second detector 21 projection data may advantageously be reduced.

The resultant combined projection data is reconstructed by the combined data reconstructor 310. Again, the reconstruction may be performed using filtered back projection techniques as are well known to those skilled in the art, although iterative or other suitable reconstruction techniques may also be implemented. As the projection data generated by the second detector 21 is typically of relatively high resolution, the reconstruction parameters may be established accordingly. In this regard, it should be noted that the relatively smaller projection matrices resulting from the use of relatively smaller regions of interest can reduce the reconstruction time of an iterative reconstruction, thus improving the attractiveness of iterative techniques.

In one implementation, the various functions described above are implemented via computer readable instructions stored on a disk, memory, or other storage media which are executed by one or more of the computer processors associated with the reconstructor 26. Moreover, some of the functionality, such as that provided by the first data reconstructor and the combined data reconstructor, may be performed using common functions or routines which are executed as desired.

In operation, and with reference to FIG. 5, scan data is obtained at step 502, for example by conducting a CT scan of the object using a scanner 10 such as the one illustrated in FIG. 1.

At 504, the projection data from the first detector is reconstructed to generate the first volumetric image data 312.

At 506, the ROI is identified.

At 508, the ROI data is removed or filtered from the first volumetric image data so as to generate the modified image data 316.

At 510, the forward projections corresponding to the second detector 21 trajectory are calculated.

At 512, the projection data from the second detector 21 is combined with the calculated projection data.

At 514, the resultant projection data is reconstructed to generate the volumetric image data 318.

At 516, a human readable image indicative of the volumetric image data is generated and displayed, for example on a monitor associated with the operator console 44. As is well known in the art, the human readable image(s) can take various forms, for example including one or more image slices, volume rendered images, or the like.

In this regard, it should be noted that steps 508, 510, and 512 need not be performed in temporal sequence for all projections in the data set. More particularly, the forward projections may be identified and calculated, the ROI removed, and the projection data combined on a projection-by-projection basis, with the process repeated for each desired projection. Moreover, the projections may be performed retrospectively using previously acquired data.

Other variations are also possible. As illustrated in FIG. 1, the first 20 and second 21 detectors and the respective x-ray sources 12, 13 are angularly offset by approximately 90°, thus generating additional data as compared to a scanner having only a single detector. The data from the first 20 and second 21 detectors may be combined to generate volumetric data having a relatively higher temporal resolution, for example in the imaging of cyclically moving objects such as the heart.

At step 602, combined projection data is generated as described above in relation to FIGS. 3 and 5, with the region in the vicinity of the heart selected as the ROI. As will be appreciated, two projection data sets are thus available: the projection data generated by the first detector 20, and the combined projection data.

At step 604, temporally corresponding projections, such as those obtained at a desired cardiac phase, are selected from the respective data sets.

At step 606, the selected projections are reconstructed to generate volumetric data indicative of the desired cardiac phase.

At step 608, the process is repeated as desired for additional cardiac phases.

The desired human readable image(s) are generated and displayed as desired at step 610.

Where the detectors 20, 21 are offset by 90°, the temporal resolution is improved by about a factor of two over that obtained using a scanner having a single detector. Where the second detector 21 has a relatively higher spatial resolution than that of the first detector 20, the reconstructed image also has a higher resolution than would be obtained in a scanner having a second detector which has a spatial resolution similar to that of the first detector 20. Moreover, the improved temporal resolution allows the use of a narrower gating window, thereby reducing blunting in the reconstructed image.

Information from the first 20 and second 21 detectors may also be used in multiple energy or spectral imaging in which spectrally coded projections are generated. Such projections are typically generated by varying the x-ray source 12, 13 voltage from view to view, or through the use of spectral or energy resolving detectors which provide outputs indicative of radiation detected in more than one energy range.

In particular, the spectral information can be used to differentiate between multiple material base functions. In one implementation, both the first 20 and second 21 detectors produce spectrally coded data. In another, only one of the detectors 20, 21 provides spectral information. In any case, the material base functions may be separated in an optimal or otherwise desired fashion.

As one example, a soft tissue region is selected as the ROT. The first detector 20 works with a spectral coding that provides separation between bone and soft tissue base functions. The second detector 21 may be optimized for another contrast, for example, the separation of contrast agent and soft tissue base functions. As the base functions include the energy dependence of the line integrals, the measurement from the first detector 20 can be used to process the second detector 21 measurements. Moreover, the subtraction of bone from the second detector 21 measurements can be used to reduce artifacts which would otherwise appear in the reconstruction, particularly where strong absorption gradients are located near the ROI 314.

As still another variation, the ROT 314 may be selected interactively. With reference to FIG. 7, a scout or other low resolution scan is obtained at step 702. At 704, the scout scan is displayed, for example on the monitor associated with the operator console 44. At 706, the operator identifies the desired ROT 304. The controller 28 uses this information to adjust the position of the object support 16 so that the ROT is located, and ideally centered, in the field of view of the second detector 21. The object is then scanned at 710, and the data is processed as describe above at 712. As the field of view of the second detector 21 is typically centered at the center of rotation of the gantry 18, such a procedure is especially usefully in situations where the ROT is offset from the center of the object under examination or otherwise relatively large in relation to the field of view of the second detector 21.

It should also be noted that the detectors 20, 21 and their respective sources may be offset by angles other than 90°. Moreover, one of the x-ray sources may be omitted, with the second detector 21 being centered in otherwise partially angularly coextensive with the first detector 20. In such an implementation, the data from both the first 20 and second 21 detectors is be used to generate the first volumetric data 312. Further processing of the projection data would then occur as described above.

Of course, modifications and alterations will occur to others upon reading and understanding the preceding description. It is intended that the invention be construed as including all such modifications and alterations insofar as they come within the scope of the appended claims or the equivalents thereof.

1. A tomographic apparatus comprising:
a first radiation sensitive detector which generates first projection data indicative of an object disposed in an examination region;
a second radiation sensitive detector which generates second projection data indicative of the object, wherein the second detector has a second transaxial field of view, and wherein the second transaxial view is smaller than a transaxial dimension of the object, whereby volumetric data reconstructed using the second projection data would contain truncation artifacts;
means for correcting the second projection data so as to reduce the truncation artifacts, wherein the correction is a function of the first projection data;
a corrected data reconstructor which generates volumetric data indicative of the corrected second projection data.

2. The apparatus of claim 1 wherein the first detector has a first transaxial field of view and a first resolution, wherein the second detector has a second resolution, wherein the second transaxial field of view is smaller than the first transaxial field of view, and wherein the second resolution is greater than the first resolution.

3. The apparatus of claim 2 wherein the object includes a beating heart, the apparatus includes means for selecting projections from the first projection data and the corrected second projection data as a function of the cardiac phase, and the corrected data reconstructor generates volumetric data indicative of the selected projections.

4. The apparatus of claim 2 wherein the first detector includes an arcuate x-ray detector and wherein the second detector includes a flat panel x-ray detector.

5. The apparatus of claim 1 including:
a first reconstructor which generates first volumetric data indicative of the first projection data;
an ROI filter which filters the first volumetric data so as to remove an ROI therefrom;
a projection calculator which calculates a plurality of projections through the filtered first volumetric data; and
a projection combiner which combines the projection data and the calculated projections.

6. The apparatus of claim 5 wherein the projection combiner subtracts a calculated projection from a spatially corresponding projection of the second projection data.

7. The apparatus of claim 5 including means for identifying the ROI.

8. The apparatus of claim 7 wherein the ROI is identified using a segmentation technique.

9. The apparatus of claim 1 wherein the first and second detectors are x-ray detectors and wherein the apparatus includes a first x-ray source disposed across the examination region from the first detector and a second x-ray source disposed across the examination region form the second detector.

10. A tomography method comprising:
receiving first projection data generated by a first radiation sensitive detector, wherein the projection data is indicative of an interior of an object, and wherein the object has a transaxial dimension;
receiving second projection data generated by a second radiation sensitive detector, wherein the second projection data is indicative of the interior of the object, wherein the second detector has a second transaxial field of view, and wherein the second transaxial view is smaller than a transaxial dimension of the object, whereby volumetric data reconstructed using the second projection data would contain truncation artifacts;
correcting the second projection data so as to reduce the truncation artifacts, wherein the correction is a function of the first projection data;
reconstructing the corrected second projection data;
generating a human readable image indicative of the reconstructed data.

11. The method of claim 10 including reconstructing the first projection data to generate first volumetric data, and wherein the correction is a function of the first volumetric data.

12. The method of claim 11 including calculating a plurality of first projections through the first volumetric data.

13. The method of claim 12 wherein calculating a plurality of first projections includes interpolating the first volumetric data, and wherein the interpolation is a high order interpolation.

14. The method of claim 12 including identifying a region of interest in the first volumetric data and wherein the first projections do not include a contribution from the region interest.

15. The method of claim 14 wherein the second projection data includes a plurality of second projections and including:
subtracting a calculated projection from a second projection;
repeating the step of subtracting for each of a plurality of second projections.

16. The method of claim 11 wherein the first projection data includes a plurality of projections and wherein reconstructing includes:
selecting temporally corresponding projections in the first projection data and the corrected second projection data; and
reconstructing the selected projections.

17. The method of claim 16 wherein the object includes a beating heart and wherein selecting includes selecting the projections as a function of the cardiac phase.

18. The method of claim 10 wherein the second detector has a transaxial resolution which is higher than a transaxial resolution of the first detector.

19. The method of claim 18 wherein the first detector includes a plurality of radiation sensitive detector elements disposed in an arc about the examination region.

20. The method of claim 18 including using spectral information to differentiate between material base functions.

21. A computer readable storage medium containing instructions which, when executed by a computer, cause the computer to carry out a method for reducing truncation artifacts resulting from the tomographic reconstruction of projection data acquired using a first x-ray detector having a first transaxial field of view less than a transaxial dimension of an object under examination, wherein the projection data includes projections which include a contribution from a portion of the object located inside the transaxial field of view and a contribution from a portion of the object located outside the transaxial field of view, the method comprising:
using first volumetric data indicative of a measured radiation attenuation of the object to modify a projection so as to reduce a contribution to the projection from a portion of the object located outside the transaxial field of view;
repeating the step of using volumetric data for each of a plurality of projections;
reconstructing the modified projections to generate second volumetric data indicative of the radiation attenuation of the object.

22. The computer readable storage medium of claim 21 wherein the method includes:
reconstructing projection data acquired using a second x-ray detector having a second transaxial field of view larger than the transaxial dimensions of the object so as to generate the first volumetric data.

23. The computer readable storage medium of claim 22 wherein the method includes identifying a region of interest of the object, which region of interest is located inside the first transaxial field of view, and wherein the step of using first volumetric data includes reducing a contribution to the projection from a portion of the object located outside the region of interest.

24. The computer readable storage medium of claim 23 wherein identifying includes segmenting the first volumetric data.

25. The computer readable storage medium of claim 21 wherein the method includes:
calculating a projection through the first volumetric data;
using the calculated projection to modify a projection of the projection data.

26. The method of claim 21 wherein the first volumetric data includes a first spatial resolution and the second volumetric data includes a second spatial resolution, and wherein the second spatial resolution is higher than the first spatial resolution.

27. The method of claim 21 including using spectral information to differentiate between material base functions.

28. A computed tomography apparatus comprising:
a first x-ray source;
a first x-ray detector which receives x-ray generated by the first x-ray source and which have traversed an examination region, wherein the first x-ray detector has a first transaxial field of view and a first transaxial resolution;
a second x-ray source;
a second x-ray detector which generates a plurality of projections indicative of x-rays generated by the second x-ray source and which have traversed the examination region, wherein the second x-ray detector has a second transaxial field of view and a second transaxial resolution, and wherein the first transaxial field of view is larger than the second transaxial field of view and the first transaxial resolution is less than the second transaxial resolution;
a first reconstructor operatively connected to the first x-ray sensitive detector and adapted to generate first volumetric data;
an ROI filter (304) which filters an ROI from the first volumetric data;
a projection calculator which calculates projections through the filtered first volumetric data;
a projection data subtractor which subtracts the calculated projections from spatially corresponding projections from the second x-ray detector;
a data reconstructor which generates volumetric data indicative of the subtracted projection data.
